(19) United States
(12) Patent Application Publication
(10) Pub. No.: US 2022/0413386 A1
(43) Pub. Date: Dec. 29, 2022

(54) Improved Transparency in Negative Epoxy Photoresist

(71) Applicants: Jeremy Golden, Burlington, MA (US); Gerard Gomes, West Boylston, MA (US); Michael Cronin, Norfolk, MA (US)

(72) Inventors: Jeremy Golden, Burlington, MA (US); Gerard Gomes, West Boylston, MA (US); Michael Cronin, Norfolk, MA (US)

(21) Appl. No.: 17/748,876

(22) Filed: May 19, 2022

Related U.S. Application Data
(60) Provisional application No. 63/208,569, filed on Jun. 9, 2021.

(51) Int. Cl. G03F 7/038; G03F 7/029; G03F 7/033; C07C 381/12

(52) U.S. Cl. CPC G03F 7/0382; G03F 7/029; G03F 7/033; C07C 381/12

(57) ABSTRACT

Disclosed herein is a photosensitive composition comprising an epoxy resin, a photoacid generator, and an antioxidant. The disclosed composition is useful for producing relief images having reduced yellowing or other discoloration due to thermal oxidation.

### REFERENCE TO PRIOR-FILED APPLICATION

The present application claims the benefit of U.S. Provisional Patent Application No. 63/208,569, by Golden, et al., filed 9 Jun. 2021, entitled Improved transparency in Negative Epoxy Photoresist, which application is incorporated herein by reference.

### FIELD OF THE INVENTION

The present application for patent relates to improvements in negative-working photoresists, especially those having improved transparency characteristics.

### BACKGROUND

Photoimageable epoxies have been used in many applications: MEMS (Micro Electo Mechanical Systems), Microfluidics such as Piezo Ink-jet, Sensors, Light Waveguides, Lenses, LED, MicroLED, labs on a chip and many other applications. These photoresists are used as permanent devices, rather than the more traditional photoresist, which is sacrificial.

The permanent epoxy photoresist films darken (yellow/brown) over time with thermally induced oxidation; this is a known issue with SU-8 type resists and cured epoxy materials generally. The darkening reduces optical transparency across a wide range of wavelengths from UV-IR. This darkening limits its widespread use in optical applications such as waveguides, LED, micro LED, sensors, and the like.

Photolytic quenchers, such as hindered amine light stabilizers (HALS), such as derivatives of 2,2,6,6-tetramethylpiperidine, which preventatively absorb UV energy to protect the film from yellowing have been tried without success. While HALS quenchers appear to be useful in reducing yellowing from photolytic stresses, they are less effective in reducing yellowing thought to be caused by thermal oxidation, Moreover, HALS quenchers are basic and can absorb acids, thus reducing the curing effectiveness of acid catalyzed epoxy resins such as those used in epoxy based photoresists.

Therefore there remains a need for a photoimagable epoxy-based material having a suitable free radical quencher, chosen such that is can protect against darkening while not interfering with the lithographic performance of a high aspect ratio epoxy photoresist.

### BRIEF DESCRIPTION OF THE FIGURES

FIG. 1 shows ultraviolet-visible (UV-Vis) percent transmission vs. wavelength curves for the photoresist material, prepared and thermally treated as described in Example 1.

FIG. 2 shows UV-Vis percent transmission vs. wavelength curves for the photoresist material, prepared and thermally treated as described in Example 2.

FIG. 3 shows UV-Vis percent transmission vs. wavelength curves for the photoresist material, prepared and thermally treated as described in Example 3

FIG. 4 shows a lithographic comparison between the resist material of Example 1 and the resist material of Example 2.

l FIG. 5 shows UV-Vis percent transmission vs. wavelength curves, comparing a commercially available photoresist material, HARE SQ25, from KemLab, Inc., with the same photoresist, but having the radical quencher of Example 1, prepared and thermally treated as described.

### DETAILED DESCRIPTION

As used herein, the conjunction “and” is intended to be inclusive and the conjunction “or” is not intended to be exclusive unless otherwise indicated. For example, the phrase “or, alternatively” is intended to be exclusive. The conjunction “or” may also be exclusive when required by context. As used herein, novolak polymers are condensation products of phenolic compounds and aldehydes such as formaldehyde, acetaldehyde or substituted or unsubstituted benzaldehydes or condensation products of phenolic compounds and substituted or unsubstituted methylol compounds. As used herein, the terms “photoinitiator” and “photoacid generator” are intended to be synonymous. As used herein, the “exposure dose” or “dose” is understood to denote the energy per square length impinging on a workpiece such as a photoresist coating unless otherwise specified. Exposure dose units are usually expressed as mJ/cm². As used herein, it is understood that applied temperature numbers refer to the set-point or measured temperature of the heating device, not the temperature of any workpiece unless explicitly noted. As used herein, epoxy resins are understood to be polyethers or polyesters of polyhydric resins, wherein the ether or ester comprises an epoxy group. As used herein, phenolic epoxy resins are understood to be epoxy resins that are polyethers of polyhydric phenolic resins, wherein the ether comprises an epoxy group, such as, for example, in polyglycidyl polyethers of phenolic resins.

Disclosed herein is a photosensitive composition, comprising: (a) an epoxy resin; (b) a photoacid generator; and (c) an antioxidant.

Non limiting examples of epoxy resins include bisphenol A-based epoxy compounds, wherein the two phenyl rings in each oligomeric or polymeric unit are joined by a -propane-2,2-diyl- group, such as EPON™ Resin SU-8, commercially available from Hexion, Inc. of Columbus Ohio, having idealized structure (I). Further non limiting examples of epoxy resins include bisphenol F-based epoxy compounds, wherein the -propane-2,2-diyl- group of the bisphenol A-based resin is replaced by a methylene group, bisphenol S-based epoxy compounds, wherein the -propane-2,2-diyl-group of the bisphenol A-based resin is replaced by a sulfonyl group, novolak resin-based epoxy compounds, resol resin-based epoxy compounds, and poly (hydroxystyrene)-based epoxy compounds. The epoxy resins may be fully or partially substituted with epoxy groups. In the negative-working photosensitive compositions disclosed herein, epoxy resins may be present from 10%-75% of the photosensitive composition, including solvents. The amount of epoxy resin present in a given formulation is not contemplated to be limiting and depends on the desired film thickness, as an example, in certain thick film applications, the epoxy resin may be present at between 55% and 65% of the photosensitive composition, including solvents.

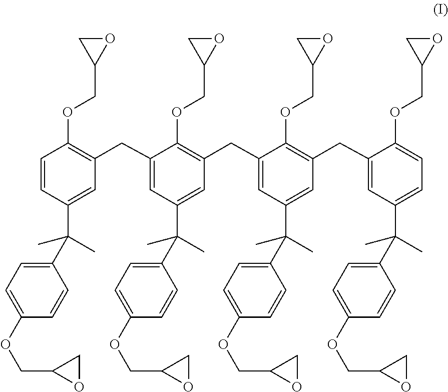 [SMILES: CC(C)(c1ccc(OCC2CO2)cc1)c1ccc(OCC2CO2)c(Cc2cc(C(C)(C)c3ccc(OCC4CO4)cc3)cc(Cc3cc(C(C)(C)c4ccc(OCC5CO5)cc4)cc(Cc4cc(C(C)(C)c5ccc(OCC6CO6)cc5)ccc4OCC4CO4)c3OCC3CO3)c2OCC2CO2)c1]

Photoacid generators are present to generate acid upon exposure to actinic irradiation. Such photoacid generators can be present alone or in admixture and may be selected in accordance with their ability to generate acid using radiation of a particular wavelength, wavelength range, energy or energy range. Without intending to be bound by theory, it is thought that photoacid generators produce acids by different mechanisms. Accordingly, photoacid generators may be present at selected concentrations, alone or in admixture, to optimize acid output for the selected form of actinic irradiation.

For example, photoacid generators may be present in the solid resist film at concentrations from about 1 mole/g of resist solids to about 1,000 mole/g (micromoles per gram) of resist solids. As a further example, photoacid generators may be present in the solid resist film at concentrations from about 5 mole/g of resist solids to about 300 mole/g of resist solids. As a still further example, photoacid generators may be present in the solid resist film at concentrations from about 10 mole/g of resist solids to about 150 mole/g of resist solids. As a still further example, photoacid generators may be present in the solid resist film at concentrations from about 15 mole/g of resist solids to about 100 mole/g of resist solids. Alternatively, for example, photoacid generators may be present as 0.1%-15% of total resist solids. As a further example, PAGs may be present as 1%-10% of total resist solids.

Photoacid generators may have different chemical compositions. For example, without limitation, suitable photoacid generators may be onium salts, dicarboximidyl sulfonate esters, oxime sulfonate esters, diazo(sulfonyl methyl) compounds, disulfonyl methylene hydrazine compounds, nitrobenzyl sulfonate esters, biimidazole compounds, diazomethane derivatives, glyoxime derivatives, (3-ketosulfone derivatives, disulfone derivatives, nitrobenzylsulfonate derivatives, sulfonic acid ester derivatives, imidoyl sulfonate derivatives, halogenated triazine compounds, equivalents thereof or combinations thereof.

Onium salt photoacid generators may comprise, without limitation, alkyl sulfonate anions, substituted and unsubstituted aryl sulfonate anions, fluoroalkyl sulfonate anions, fluoarylalkyl sulfonate anions, fluorinated arylalkyl sulfonate anions, hexafluorophosphate anions, hexafluoroarsenate anions, hexafluoroantimonate anions, tetrafluoroborate anions, equivalents thereof or combinations thereof.

Specifically, without limitation suitable photoacid generators may include triphenylsulfonium trifluoromethanesulfonate, triphenylsulfonium nonafluoro-n-butanesulfonate, triphenylsulfonium perfluoro-n-octanesulfonate, and triphenylsulfonium 2-(bicyclo[2.2.1]heptan-2-yl)-1,1,2,2-tetrafluoroethanesulfonate, 4-cyclohexylphenyldiphenylsulfonium trifluoromethanesulfonate, 4-cyclohexylphenyldiphenylsulfonium nonafluoro-n-butanesulfonate, 4-cyclohexylphenyldiphenylsulfonium perfluoro-n-octanesulfonate, 4-cyclohexylphenyldiphenylsulfonium 2-(bicyclo[2.2.1]heptan-2-yl)-1,1,2,2-tetrafluoroethanesulfonate, 4-methanesulfonylphenyldiphenylsulfonium trifluoromethanesulfonate, 4-methanesulfonylphenyldiphenylsulfonium nonafluoro-n-butanesulfonate, 4-methanesulfonylphenyldiphenylsulfonium perfluoro-n-octanesulfonate, and 4-methanesulfonylphenyldiphenylsulfonium 2-(bicyclo[2.2.1]heptan-2-yl)-1,1,2,2-tetrafluoroethanesulfonate, diphenyliodonium trifluoromethanesulfonate, diphenyliodonium nonafluoro-n-butanesulfonate, diphenyliodonium perfluoro-n-octanesulfonate, diphenyliodonium 2-(bicyclo[2.2.1]heptan-2-yl)-1,1,2,2-tetrafluoroethanesulfonate, bis(4-t-butylphenyl)iodonium trifluoromethanesulfonate, bis(4-t-butylphenyl)iodonium nonafluoro-n-butanesulfonate, bis(4-t-butylphenyl)iodonium perfluoro-n-octanesulfonate, bis(4-t-butylphenyl)iodonium 2-(bicyclo[2.2.1]heptan-2-yl)-1,1,2,2-tetrafluoroethanesulfonate, 1-(4-n-butoxynaphthalen-1-yl)tetrahydrothiophenium trifluoromethanesulfonate, 1-(4-n-butoxynaphthalen-1-yl)tetrahydrothiophenium nonafluoro-n-butanesulfonate, 1-(4-n-butoxynaphthalen-1-yl)tetrahydrothiophenium perfluoro-n-octanesulfonate, 1-(4-n-butoxynaphthalen-1-yl)tetrahydrothiophenium 2-(bicyclo[2.2.1]heptan-2-yl)-1,1,2,2-tetrafluoroethanesulfonate, 1-(6-n-butoxynaphthalen-2-yl)tetrahydrothiophenium trifluoromethanesulfonate, 1-(6-n-butoxynaphthalen-2-yl)tetrahydrothiophenium nonafluoro-n-butanesulfonate, 1-(6-n-butoxynaphthalen-2-yl)tetrahydrothiophenium perfluoro-n-octanesulfonate, 1-(6-n-butoxynaphthalen-2-yl)tetrahydrothiophenium 2-(bicyclo[2.2.1]heptan-2-yl)-1,1,2,2-tetrafluoroethanesulfonate, 1-(3,5-dimethyl-4-hydroxyphenyl)tetrahydrothiophenium trifluoromethanesulfonate, 1-(3,5-dimethyl-4-hydroxyphenyl)tetrahydrothiophenium nonafluoro-n-butanesulfonate, 1-(3,5-dimethyl-4-hydroxyphenyl)tetrahydrothiophenium perfluoro-n-octanesulfonate, 1-(3,5-dimethyl-4-hydroxyphenyl)tetrahydrothiophenium 2-(bicyclo[2.2.1]heptan-2-yl)-1,1,2,2-tetrafluoroethanesulfonate N-(trifluoromethanesulfonyloxy)bicyclo[2.2.1]hept-5-ene-2,3-dicarboximide, N-(nonafluoro-n-butanesulfonyloxy)bicyclo[2.2.1]hept-5-ene-2,3-dicarboxyimide, N-(perfluoro-n-octanesulfonyloxy)bicyclo[2.2.1]hept-5-ene-2,3-dicarboxyimide, N-[2-(bicyclo[2.2.1]heptan-2-yl)-1,1,2,2-tetrafluoroethanesulfonyloxy]bicyclo[2.2.1] hept-5-ene-2,3-dicarboxyimide, N-[2-(tetracyclo[4.4.0.1^{2,5}1^{7,10}]dodecan-3-yl)-1,1-difluoroethanesulfonyloxy]bicyclo[2.2.1]hept-5-ene-2,3-dicarboxyimide, 1,3-dioxoisoindolin-2-yl trifluoromethanesulfonate, 1,3-dioxoisoindolin-2-yl nonafluoro-n-butane sulfonate, 1,3-dioxoisoindolin-2-yl perfluoro-n-octane sulfonate, 3-dioxoisoindolin-2-yl 2-(bicyclo[2.2.1]heptan-2-yl)-1,1,2,2-tetrafluoroethanesulfonate, 3-dioxoisoindolin-2-yl N-[2-(tetracyclo[4.4.0.1^{2,5}1^{7,10}]dodecan-3-yl)-1,1-difluoroethanesulfonate, 1,3-dioxo-1H-benzo[de]isoquinolin-2(3H)-yl trifluoromethanesulfonate, 1,3-dioxo-1H-benzo[de]isoquinolin-2(3H)-yl nonafluoro-n-butane sulfonate, 1,3-dioxo-1H-benzo[de]isoquinolin-2(3H)-ylperfluoro-n-octanesulfonate, 1,3-dioxo-1H-benzo[de]isoquinolin-2(3H)-yl2-(bicyclo[2.2.1]heptan-2-yl)-1,1,2,2-tetrafluoroethanesulfonate, or 1,3-dioxo-1H-benzo[de]isoquinolin-2(3H)-yl N-[2-(tetracyclo [4.4.0.1^{2,5}1^{7,10}]dodecan-3-yl)-1,1-difluoroethanesulfonate, (E)-2-(4-methoxystyryl)-4,6-bis(trichloromethyl)-1,3,5-triazine, 2-(Methoxyphenyl)-4,6-bis- (trichloromethyl)-s-triazine, 2-[2-(Furan-2-yl)ethenyl]-4,6-bis(trichloromethyl)-s-triazine, 2-[2-(5-methylfuran-2-yl)ethenyl) -4,6-bis(trichloromethyl)-s-triazine, 2-[2-(3,4-Dimethoxyphenyl)ethenyl]-4,6-bis(trichloromethyl)-s-triazine, equivalents thereof or combinations thereof. Suitable photoacid generators may also include onium salts comprising anions and cations in combinations not shown supra.

Photoacid generators may be selected for specific wavelength ranges. Without limitation, for example, in the near UV-visible range (300 nm-440 nm), it is known in the art that naphthalene dicarboximidyl triflate (NIT) (1,3-dioxo-1H-benzo[de]isoquinolin-2(3H)-yl trifluoromethanesulfonate) (IV) and other imide esters such as (V) and VIII) are useful. In addition, onium salts such as (III) may be used advantageously, as well as oxime sulfonic acid ester such as (II), (VI), and (VII). The abbreviation OA represents a sulfonate group having alkyl, aryl, fluoroalkyl such as —CF₃, —C₄F₉, —C₈F₁₇, or fluoroaryl substituents.

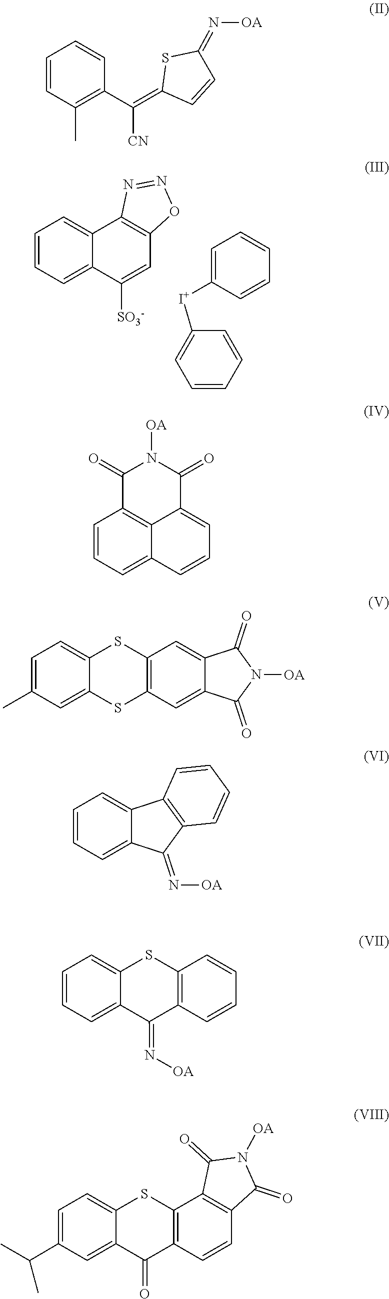

Certain suitable photoacid generators are available commercially as products whose structures are undisclosed or have disclosed generic structures comprising undisclosed mixtures. For example, the product “Photoinitiator UVI 6976,” available from Dow Chemical, or distributors such as Synasia Corporation of Metuchen, N.J., is believed to comprise a mixture of structures such as those in (IX). Further examples include Irgacure® 290, available from BASF Corporation (BASF), believed to comprise the structure (X), GSID 26, available from BASF Corporation (XI), and GSID 26-1, available from BASF, believed to comprise structure (XI). Further examples include the CPI®-100/200 Series, having generic cation structure (XII) and alternatively comprising anions PF₆⁻, SbF₆⁻, PF₃(C₂F₅)₃⁻, and B(C₆F₅)₄⁻, available from San-Apro Ltd., of Kyoto, Japan, the CPI®-300 Series having generic cation structure (XIII) and alternatively comprising anions B(C₆F₅)₄⁻, and “FG Anion,” available from San-Apro Ltd., of Kyoto, Japan, and the CPI®-400 Series having generic cation structure (XIV) and alternatively comprising anions PF₃(C₂F₅)₃⁻, and B(C₆F₅)₄, available from San-Apro Ltd., of Kyoto, Japan. In (XIII) and (XIV), R₁-R₆ are not disclosed by the manufacturer.

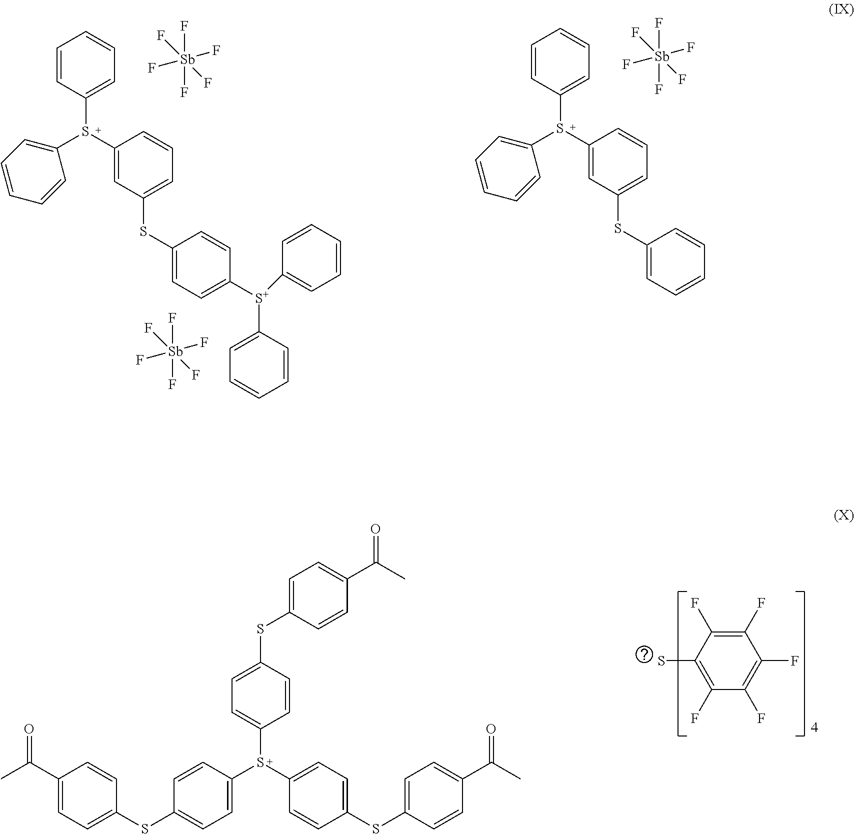

Various antioxidant free radical quenchers may be chosen to protect against darkening or discoloration, while not interfering with the lithographic performance of a high aspect ratio epoxy resist. These include, without limitation, Irganox® 1135 from BASF, believed to comprise structure (XV) and possibly similar compounds, Irganox® 1141 from BASF, believed to comprise structure (XVI) and possibly similar compounds, and Irganox® 1198 from BASF, believed to comprise structure (XVII) and possibly similar compounds.

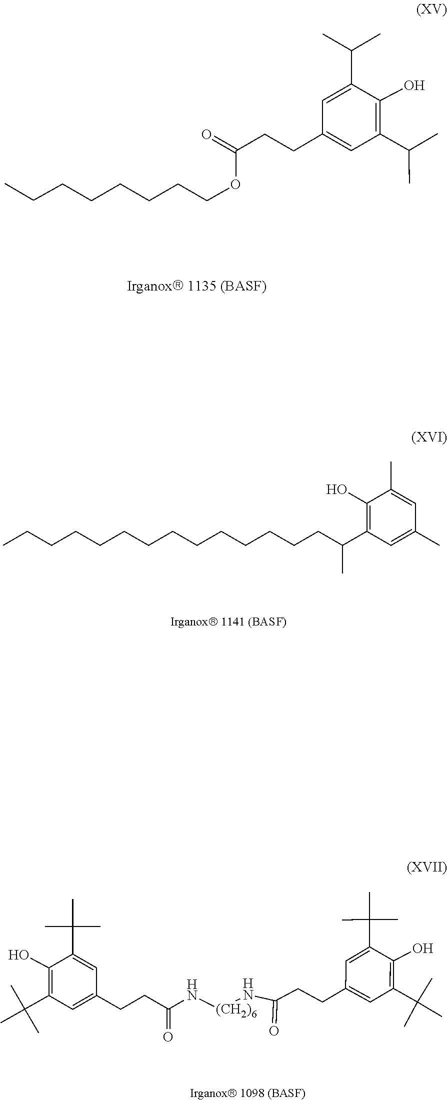

Further examples of suitable antioxidants include phenolic compounds such as 2,6-di-tert-butyl-4-methylphenol, 2-tert-butyl-4,6-dimethylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, nonylphenols which are linear or branched in the side chains, for example 2,6-di-nonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1′-yl)phenol, 2,4-dioctylthiomethyl-6-tert-butylphenol, 2,4-dioctylthiomethyl-6-methylphenol, 2,4-dioctylthiomethyl-6-ethylphenol, 2,6-di-dodecylthiomethyl-4-nonylphenol, 2,6-di-tert-butyl-4-methoxyphenol, 2,5-di-tert-butylhydroquinone, 2,5-di-tert-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis(3,5-di-tert-butyl-4-hydroxyphenyl) adipate, 2,2′-methylenebis(6-tert-butyl-4-methylphenol), 2,2′-methylenebis(6-tert-butyl-4-ethylphenol), 2,2′-methylenebis[4-methyl-6-(α-methylcyclohexyl)phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis [6-(α-methylbenzyl)-4-nonylphenol], 2,2′-methylenebis [6-(α, α-dimethylbenzyl)-4-nonylphenol], 4,4′-methylenebis (2,6-di-tert-butylphenol), 4,4′-methylenebis(6-tert-butyl-2-methylphenol), 1,1-bis(5-tert-butyl-4-hydroxy-2-methylphenyl)butane, 2,6-bis (3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methylphenyl)-3-n-dodecylme rcaptobutane, ethylene glycol bis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy- 5′-methylbenzyl)-6-tert-butyl-4-methylphenyl]terephthalate, 1,1-bis-(3,5-dimethyl-2-hydroxyphenyl)butane, 2,2-bis(3,5-di-tert-butyl-4-hydroxyphenyl)propane, 2,2-bis-(5-tert-butyl-4-hydroxy2-methylphenyl)-4-n-dodecylme rcaptobutane, 1,1,5,5-tetra(5-tert-butyl-4-hydroxy-2-methylphenyl)pentane, 1,3,5-tris(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, 2,4,6-tris(3,5-di-tert-butyl-4-hydroxybenzyl)phenol, esters of β-(3,5-di-tert-butyl-4-hydroxyphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, esters of β-(5-tert-butyl-4-hydroxy-3-methylphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 3,9-bis [2-{3-(3-tert-butyl-4-hydroxy-5-methylphenyl)propionyloxy}-1,1-dimethylethyl]-2,4,8,10-tetraoxaspiro[5.5]-undecane, esters of β-(3,5-dicyclohexyl-4-hydroxyphenyl)propionic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane.

The instant photosensitive composition comprises one or more antioxidant free radical quenchers in an amount sufficient reduce oxidative discoloration. For example, the instant photosensitive composition may comprise one or more antioxidant free radical quenchers in amounts between 0.1%-3.0% of total solids. As a further example, the instant photosensitive composition may comprise one or more antioxidant free radical quenchers in amounts between 0.2%-2.0% of total solids. As still a further example, the instant photosensitive composition may comprise one or more antioxidant free radical quenchers in amounts between 0.3%-1.0% of total solids.

Other optional additives, which have compatibility with and can be added to the composition disclosed and claimed herein according to need, include auxiliary resins, plasticizers, surface leveling agents and stabilizers to improve the properties of the resist layer, and the like.

The photosensitive resin composition disclosed herein may be used in the form of a solution prepared by dissolving the above described ingredients in a suitable organic solvent. Examples of suitable organic solvents include ketones such as acetone, methyl ethyl ketone, cyclohexanone, methyl isoamyl ketone, methyl amyl ketone, and the like, polyhydric alcohols and derivatives thereof such as monomethyl, monoethyl, monopropyl, monobutyl and monophenyl ethers of ethyleneglycol, ethyleneglycol monoacetate, diethyleneglycol, diethyleneglycol monoacetate, propyleneglycol, propyleneglycol monoacetate, dipropyleneglycol or dipropyleneglycol monoacetate and the like, cyclic ethers such as dioxane, tetrahydrofuran and the like, esters such as methyl lactate, ethyl lactate, methyl acetate, ethyl acetate, butyl acetate and the like and solvents having aromatic groups such as anisole, ethyl benzene, xylenes, chlorobenzene, toluene and the like. These organic solvents can be used either singly or in admixture according to need.

Solvents used in the photosensitive composition are selected to allow the formation of stable solutions or dispersions and to produce uniform coatings on the desired substrate, using techniques known in the art, such as spin-coating, spray-coating, or slit or slot-coating. Exemplary solvents for this purpose include, without limitation, gamma butyrolactone (GBL), cyclopentanone, propylene glycol monomethyl ether (PGME), methyl 3-methoxyproprionate (MMP), ethyl 3-ethoxyproprionate (EEP), propylene glycol methyl ether acetate (PGMEA), ethyl lactate, 2-heptanone, cyclohexanone, methyl ethyl ketone (MEK), and acetone.

Other optional additives, which have compatibility with and can be added to the composition disclosed and claimed herein according to need, include, without limitation, auxiliary resins, plasticizers, surface leveling agents and stabilizers to improve the properties of the resist layer, and the like.

The procedure for the preparation of a patterned resist layer by using the photosensitive composition disclosed herein can be conventional. For example, a substrate such as a semiconductor silicon wafer is evenly coated with the photosensitive composition in the form of a solution by using a suitable coating machine such as a spin-coater followed by baking in a convection oven or on a hotplate to form a resist layer which is then exposed pattern-wise to actinic radiation such as deep ultraviolet light, near ultraviolet light, or visible light emitted from low-pressure, high-pressure and ultra-high-pressure mercury lamps, arc lamps, xenon lamps and the like through a photomask bearing a desired pattern on a minifying light-projection exposure apparatus and electron beams scanned in accordance with a desired pattern to build up a latent image of the pattern in the resist layer. Thereafter, the latent image in the resist layer may optionally be baked in a convection oven or on a hotplate, developed using a suitable developer solution such as an aqueous alkaline developer such as tetramethyl ammonium hydroxide or sodium hydroxide, or an organic solvent developer such as PGMEA, and rinsed with isopropyl alcohol or the like, to yield a patterned resist layer having good fidelity to the pattern of the photomask.

Thicknesses of coated films may range from 20 nm to 100 μm. To achieve these thicknesses, a combination of different spin speeds and total solids concentrations may be employed. Depending on the size of the substrate, spin speeds of from 500 rpm to 10,000 rpm may be used. Concentration may be expressed as a percentage w/w of total solids in the photosensitive composition.

The compositions described herein may specifically be practiced without comprising colorants, such as pigments, dyes, fluorescent dyes, phosphors and phosphorescent materials, optical brighteners, downconverters, upconverters, scattering media, or other materials that may reduce transparency, in order to enable the production of transparent media such as waveguides, optical windows, lenses, antireflective coatings and the like. Notwithstanding the foregoing, it may be desirable for the instant composition to comprise colorants, such as pigments, dyes, fluorescent dyes, phosphors and phosphorescent materials, optical brighteners, downconverters, upconverters, scattering media and the like to enable fabrication of materials such as encapsulants for light-emitting-diodes, or devices such as optical diffusers, displays, sensors and the like.

### EXAMPLE 1

Formulation: Into a clean glass jar were charged 61.51 grams (g) of EPON SU-8, bisphenol A resin, 7.28 g of Cationic Photoinitiator solution (UVI 6976), mixed type triarylsulfonium hexafluoroantimonate salts (50% in propylene carbonate), 31.11 g gamma butyrolactone, and 0.33 g of the free radical quencher, Irganox® 1135. The jar containing the ingredients was rolled on a jar mill roller for several hours until all ingredients were dissolved. The resulting solution was filtered into a clean container using a glass fiber membrane filter having a pore size of 1 μm (micron).

Coating: In a standard spin-coating operation, the filtered solution was dispensed onto a transparent, optical grade, 4 inch fused silica wafer and allowed to stand for 5 sec. The spin speed was then ramped up to 2000 rpm and spun for 30 sec. The coated wafer was transferred to a 65° C. hotplate, baked for 3 min, quickly transferred to a 95° C. hotplate, baked for 7 min, and then allowed to cool to give a film thickness of 25 μm.

Exposure: The coated wafer received a flood exposure dose of 270 mJ/cm², using a broadband mercury lamp, equipped with an optical cut-off filter to eliminate exposure wavelengths below 360 nm. The exposed, coated wafer was transferred to a 65° C. hotplate, baked for 1 min, quickly transferred to a 95° C. hotplate, baked for 3 min, and then allowed to cool and stand for at least 60 min.

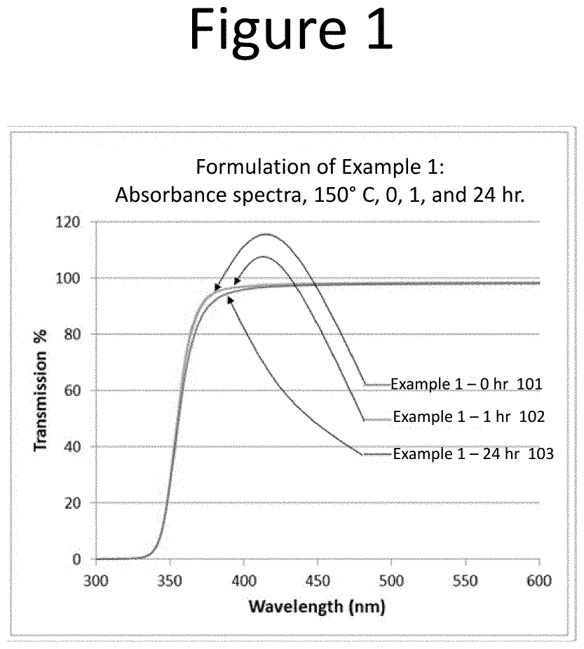

Accelerated Oxidation and Transmittance Curve Measurement: Transmittance spectra were recorded, in dual beam mode, on a Shimadzu UV-1700 UV-Vis spectrophotometer, using a matched fused silica wafer as a blank . The result 101 is shown in FIG. 1. The wafer was subjected to accelerated oxidation by baking in a 150° C. convection oven, in air, for 1 hour. The result 102 is shown in FIG. 1, wherein the transmittance curves 101 and 102 appear nearly indistinguishable from one another. The wafer was then subjected to accelerated oxidation by baking in the 150° C. convection oven, in air, for 24 hours. The result 103 is shown in FIG. 1.

### EXAMPLE 2

Similar to Example 1, except that the formulation did not include the free radical quencher, Irganox® 1135, and as described infra.

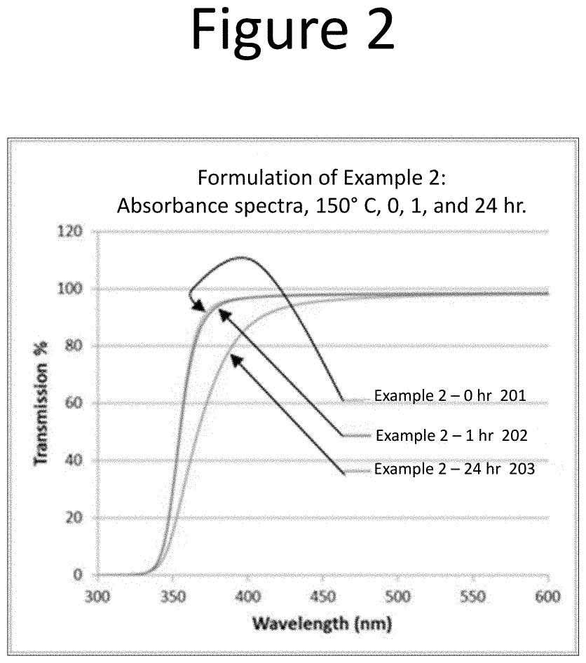

Accelerated Oxidation and Transmittance curve measurement: Transmittance spectra were recorded in dual beam mode, on a Shimadzu UV-1700 UV-Vis spectrophotometer, using a matched fused silica wafer as a blank. The result 201 is shown in FIG. 2. The wafer was subjected to accelerated oxidation by baking in a 150° C. convection oven, in air, for 1 hour. The result 202 is shown in FIG. 2, wherein the transmittance curves 201 and 202 appear to be barely distinguishable from one another. The wafer was then subjected to accelerated oxidation by baking in the 150° C. convection oven, in air, for 24 hours. The result 203 is shown in FIG. 2.

### EXAMPLE 3

Similar to Example 2, except that the formulation was the commercially available product, SU-8 2025 from Kayaku Company, which is not believed to include the free radical quencher, Irganox® 1135, and as described infra.

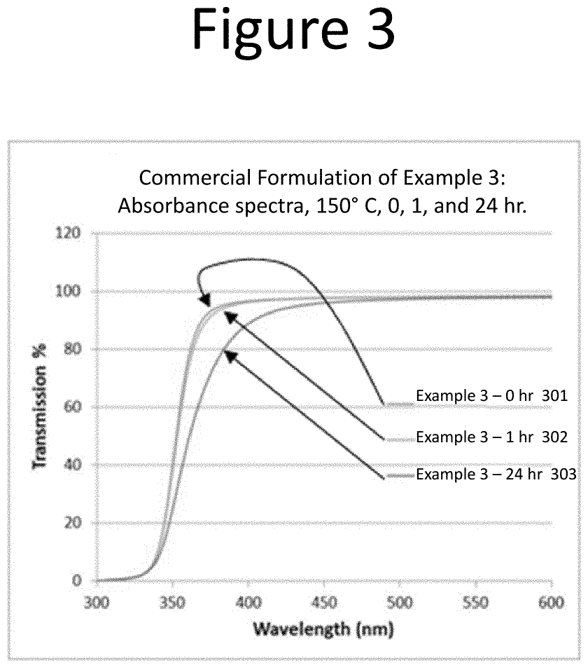

Accelerated Oxidation and Transmittance curve measurement: Transmittance spectra were recorded in dual beam mode, on a Shimadzu UV-1700 UV-Vis spectrophotometer, using a matched fused silica wafer as a blank. The result 301 is shown in FIG. 3. The wafer was subjected to accelerated oxidation by baking in a 150° C. convection oven, in air, for 1 hour. The result 30 is shown in FIG. 3, wherein the transmittance curves 301 and 30 are clearly distinguishable from one another. The wafer was then subjected to accelerated oxidation by baking in the 150° C. convection oven, in air, for 24 hours. The result 303 is shown in FIG. 3.

### Comparison of Examples 1, 2, and 3

Table 1 shows a comparison of the transmittances of the formulations of Examples 1, 2 and 3 after accelerated oxidation at 150° C. for 24 hours. As can be seen, a film made from the formulation of Example 1, comprising the free radical quencher has darkened significantly less than films made from the formulations of Example 2 and Example 3. As can be seen, the sample that comprises the free radical quencher shows significantly higher %transmission at 365 nm and at 400 nm.

TABLE 1
% Transmission after 24 hours at 150° C.

| | With free | No free | |
|---|---|---|---|
| | radical quencher | radical quencher | SU8-2025 |
| Wavelength | (Example 1) | (Example 2) | (Example 3) |
| 365 nm | 77% | 47% | 57% |
| 400 nm | 96% | 87% | 89% |

### EXAMPLE 4

In this example the lithographic properties of resists with and without the free radical quencher were compared. The formulations of Examples 1 and 2 were coated and baked as follows:

Coating: In a standard spin-coating operation, each filtered resist solution was dispensed onto a 6 in. silicon wafer and allowed to stand for 5 sec. The spin speed was then ramped up to 1000 rpm and spun for 30 sec. The coated wafer was transferred to a 65° C. hotplate, baked for 5 min, and quickly transferred to a 95° C. hotplate, baked for 15 min, and then allowed to cool to give a film thickness of 50 μm.

Exposure and Development: The wafers, coated with the formulations of Examples 1 and 2, received an exposure dose of 180 mJ/cm² through a 25 μm line and space photomask in proximity mode, using a broadband mercury lamp, equipped with an optical cut-off filter to eliminate exposure wavelengths below 360 nm. The exposed, coated wafers were transferred to a 65° C. hotplate, baked for 1 min, quickly transferred to a 95° C. hotplate, baked for 5 min, and then allowed to cool and stand for at least 60 min. The wafers were then placed in SQ developer, available from KemLab corporation, allowed to develop for 6 min, rinsed with isopropyl alcohol and dried.

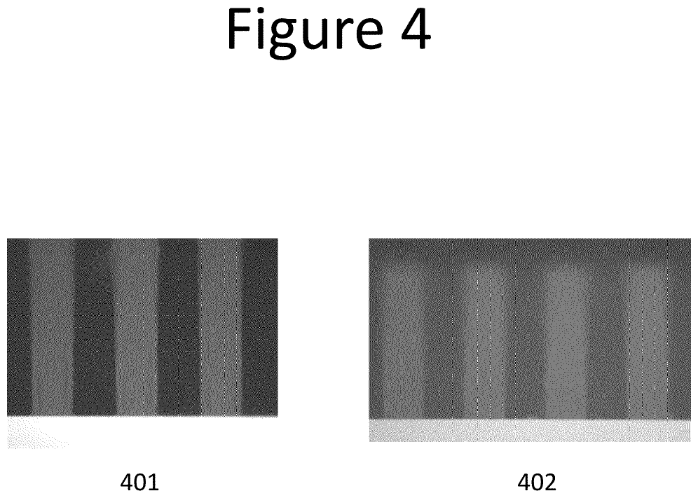

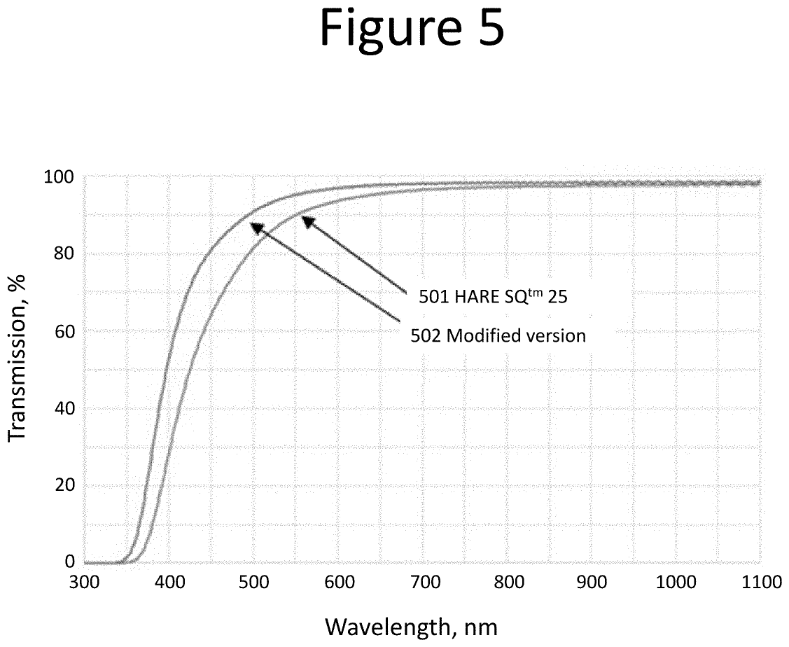

FIG. 4 shows the comparison of the lithographic features, printed into films made with the formulations of Examples 1 and 2, respectively. The printed film, made with the formulation of Example 1, is shown at 401, while the printed film, made with the formulation of Example 2, is shown at 402. No significant difference in lithographic features can be discerned.

### EXAMPLE 5

In this example, a commercially available epoxy photoresist, HARE SQ™ 25 available from KemLab Inc. was tested with and without the free radical quencher for comparison to show efficacy under accelerated thermal stress.

Formulation: Into a clean glass jar were charged HARE SQ^{tm} 25, having 61.51 grams (g) of EPON SU-8, bisphenol A resin, and 0.33 g of the free radical quencher, Irganox® 1135. The jar containing the ingredients was rolled on a jar mill roller for several hours until all ingredients were dissolved. The resulting solution was filtered into a clean container using a glass fiber membrane filter having a pore size of 1 μm to produce a modified version of the commercial product. The control sample of HARE SQ^{tm} 25 was used as provided.

Coating: In a standard spin-coating operation, the filtered solution and the control sample were dispensed onto a fused silica wafer and allowed to stand for 5 sec, the spin speed was then ramped up to 2000 rpm and spun for 30 sec. The coated wafer was transferred to a 65° C. hotplate, baked for 3 min, quickly transferred to a 95° C. hotplate, baked for 7 min, and then allowed to cool to give a film thickness of 25 μm.

Exposure: The wafers, coated with the modified version and the control, received a flood exposure dose of 180 mJ/cm², using a broadband mercury lamp, equipped with an optical cut-off filter to eliminate exposure wavelengths below 360 nm. The exposed, coated wafers were transferred to a 65° C. hotplate, baked for 1 min, quickly transferred to a 95° C. hotplate, baked for 3 min, and then allowed to cool and stand for at least 60 min. Transmittance spectra were recorded in dual beam mode, on a Shimadzu UV-1700 UV-Vis spectrophotometer, using a matched fused silica wafer as a blank.

Accelerated Oxidation and Transmittance curve measurement: The wafer was subjected to accelerated oxidation by baking on a 180° C. hotplate, in air, for 1 hour, and then on a 265° C. hotplate in air for 24 min. The result is shown in FIG. 5, wherein the transmittance curves 501 and 502 refer to the HARE SQ™ 25 control sample and the modified version, respectively. The modified version is seen to show considerably less yellowing.

Although the present invention has been shown and described with reference to particular examples, various changes and modifications which are obvious to persons skilled in the art to which the invention pertains are deemed to lie within the spirit, scope and contemplation of the subject matter set forth in the appended claims.

What is claimed is:

1. A photosensitive composition comprising:
a. an epoxy resin;
b. a photoacid generator;
c. an antioxidant.

2. The photosensitive composition of claim 1, wherein the antioxidant comprises a phenolic compound.

3. The photosensitive composition of claim 1, wherein the epoxy resin is a phenolic epoxy resin.

4. The photosensitive composition of claim 1, wherein the antioxidant comprises a phenolic compound, having a structure chosen from (XV), (XVI), and (XVII)

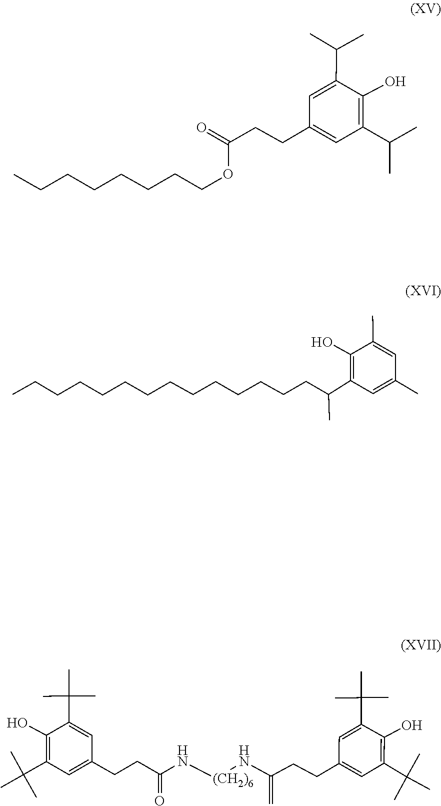

5. The photosensitive composition of claim 1, wherein the antioxidant comprises an ester or an amide of 3-(4-hydroxy-3,5-diisopropylphenyl)propanoic acid.

6. The photosensitive composition of claim 1, wherein the antioxidant comprises an ester or an amide of 2-(4-hydroxy-3,5-diisopropylphenyl)acetic acid.

7. The photosensitive composition of claim 1, wherein the antioxidant comprises an ester or an amide of 3-(3,5-di-tert-butyl-4-hydroxyphenyl)propanoic acid.

8. The photosensitive composition of claim 1, wherein the antioxidant comprises an ester or an amide of 3-(3,5-di-tert-butyl-4-hydroxyphenyl)propanoic acid.

9. The photosensitive composition of claim 1, wherein the antioxidant comprises an ester or an amide of 2-(3,5-di-tert-butyl-4-hydroxyphenyl)acetic acid.

10. The photosensitive composition of claim 1, wherein the photoacid generator comprises an onium salt.

11. The photosensitive composition of claim 1, wherein the antioxidant does not comprise a hindered amine light stabilizer.

12. The photosensitive composition of claim 1, further comprising one or more solvents, chosen from gamma butyrolactone, cyclopentanone, propylene glycol monomethyl ether. methyl 3-methoxyproprionate, ethyl 3-ethoxyproprionate, propylene glycol methyl ether acetate, ethyl lactate, 2-heptanone, cyclohexanone, methyl ethyl ketone, and acetone.

13. The photosensitive composition of claim 1, wherein the epoxy resin is chosen from a bisphenol-A-based epoxy resin, a bisphenol F-based epoxy resin, a bisphenol S-based epoxy resin, an epoxy novolak resin, an epoxy resol resin, and an epoxy poly (hydroxystyrene) resin.

14. The photosensitive composition of claim 1, wherein the photoacid generator comprises one or more sulfonium salts or one or more iodonium salts.

15. The photosensitive composition of claim 1, wherein the photoacid generator is a sulfonium salt chosen from (IX), (X), and (XI).

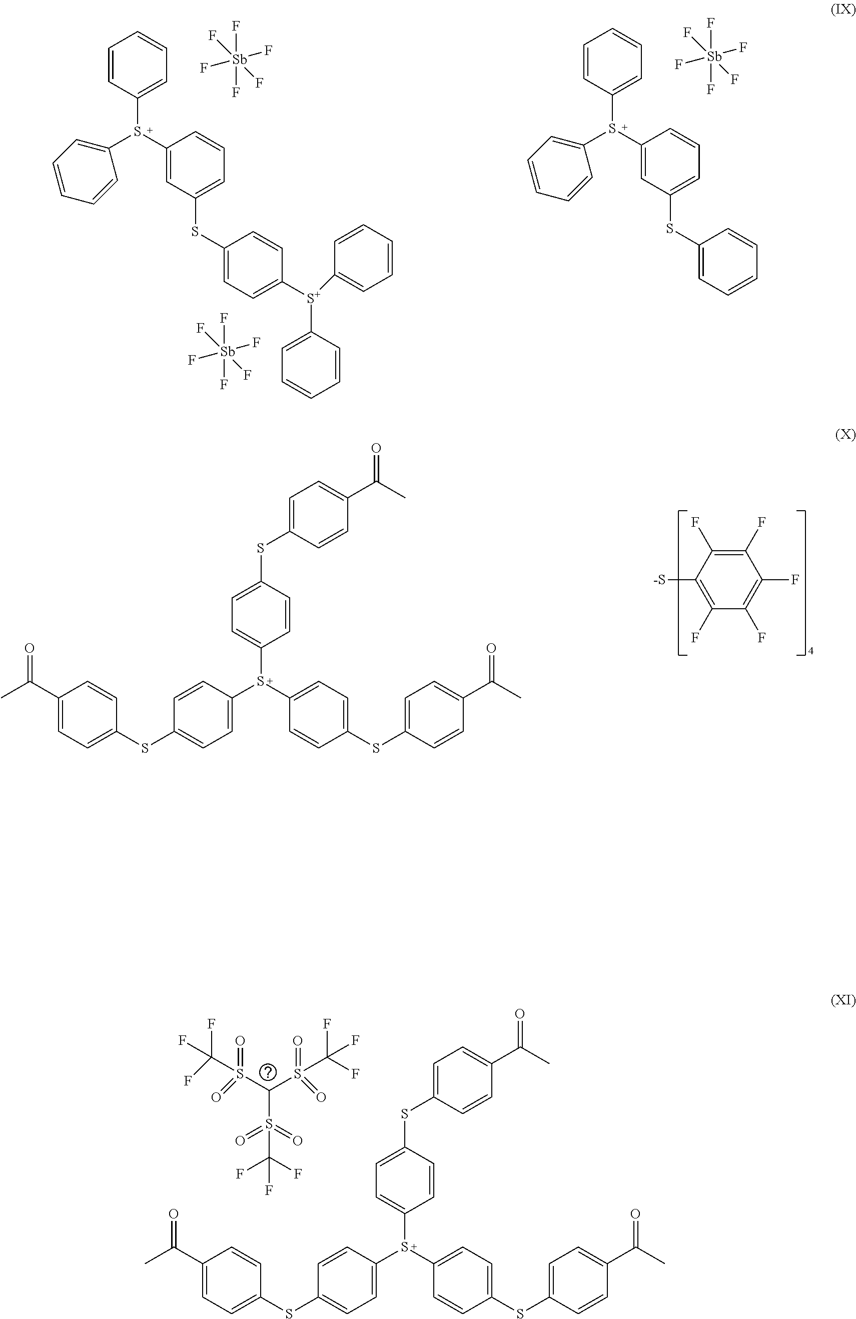

16. The photosensitive composition of claim 1, wherein the photoacid generator comprises a sulfonium cation having structure (XII), and one or more anions chosen from PF₆⁻, SbF₆⁻, PF₃(C₂F₅)₃⁻, and B(C₆F₅)₄⁻, 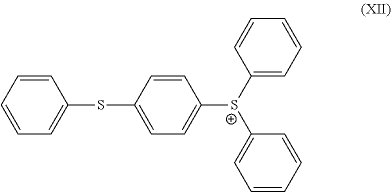 [SMILES: c1ccc(Sc2ccc([S+](c3ccccc3)c3ccccc3)cc2)cc1]
